(19) United States
(12) Patent Application Publication
(10) Pub. No.: US 2003/0109787 A1
(43) Pub. Date: Jun. 12, 2003

(54) Multiple laser diagnostics

(76) Inventor: Michael Black, Foster City, CA (US)

Correspondence Address: MAYNARD A. HOWE, RELIANT TECHNOLOGIES, INC., 205 SOUTH HELIX, SUITE 72, SOLANA BEACH, CA, 92075, US

(21) Appl. No.: 10/020,270

(22) Filed: Dec. 12, 2001

(51) Int. Cl. A61B 6/00

(52) U.S. Cl. 600/473

(57) ABSTRACT

A multiple laser diagnostic apparatus and method is provided that includes two or more lasers. Each laser simultaneously delivers a laser diagnostic beam. Each laser diagnostic beam has at least one distinct laser beam parameter. Different means to select two or more laser diagnostic beams and laser beam parameters are included. The laser diagnostic beams are combined in a combined diagnostic beam. The combined diagnostic beam is delivered at a substance at which the substance undergoes fluorescence emission. The fluorescence emission is detected and diagnostic data is analyzed or computed. The diagnostic data is displayed using displaying means. A computer program is provided to control and manage the simultaneous delivery of multiple laser diagnostic beams. Furthermore, a database is provided that contains of a plurality of laser diagnostic plans.

### CROSS-REFERENCE TO RELATED APPLICATIONS

This application is cross-referenced to copending US patent application entitled “Multiple laser treatment” by inventor Michael Black with filing date Dec. 12, 2001, which is hereby incorporated by reference.

### FIELD OF THE INVENTION

This invention relates generally to laser systems. More particularly, the present invention relates to multiple laser diagnostic systems.

### BACKGROUND

Lasers have many useful applications in the diagnoses of surfaces and materials to determine a variety of physical parameters. Lasers have also become valuable medical diagnostic instruments and are, for example, used in medical testing, drug discovery, biotechnology and imaging. According to the various types of applications, the prior art teaches different ways of utilizing laser systems to diagnose tissue that is useful for a particular application (see, for example, U.S. Pat. Nos. 5,106,387, 5,419,323, 5,562,100, 6,208,886 and 6,293,911). For example, in medical testing, catheters employing optical fibers for the illumination, viewing and treatment of tissue are used with sources of laser radiation for a variety of medical applications. Through the insertion of the catheter into a human artery or bodily cavity, laser radiation of a given wavelength can be used to illuminate tissue within the body such that the tissue fluoresces. One or more of the optical fibers conveys radiation generated by the tissue to the proximal end of the catheter where it can be analyzed to yield information about the tissue under examination. As an example to illustrate the usefulness of a catheter application in medical testing, fluorescence spectroscopy can be used to differentiate the fluorescence spectra of normal aorta and fibrous plaque to diagnose the presence of atherosclerosis in a human artery wall. Other prior art teaches laser systems capable of delivering a laser treatment beam and a diagnostic laser beam (see, for example, U.S. Pat. Nos. 4,556,057 and 4,641,650). U.S. Pat. No. 4,556,057 teaches a laser system wherein a laser treatment beam and a diagnostic laser beam are delivered independently to a tissue. U.S. Pat. No. 4,641,650 teaches a laser system wherein a diagnostic light source is used to determine the need for providing or continuing to provide laser treatment with a laser treatment beam. Although, the field of laser-induced fluorescence is expanding at an ever-increasing rate, prior art solutions and methods are teaching a single laser system to deliver a single laser beam with a particular wavelength for a diagnosis. However, there is a need to develop advanced laser diagnostic systems that are capable of diagnosing tissue or structures with the greatest variety and flexibility as new technology becomes available and new applications for lasers are discovered.

### SUMMARY OF THE INVENTION

The present invention provides a multiple laser diagnostic apparatus and method that overcomes the limitations of prior art developments and methods. The present invention provides a versatile and flexible system that meets the current needs of laser diagnostics with the greatest variety.

A multiple laser diagnostic apparatus and method of the present invention includes n lasers. Each laser simultaneously delivers a laser diagnostic beam. Each laser diagnostic beam has at least one distinct laser beam parameter. Each laser beam parameter is selected for a diagnosis. In general, the present invention includes two or more lasers. The lasers can be can be different lasers or the same type of lasers. In case of the same type of laser at least one laser beam parameter in each laser diagnostic beam is different. In general, one or more laser beam parameters of the laser diagnostic beams are different. However, one or more laser beam parameters of the laser diagnostic beams can also be the identical.

Examples of laser beam parameters are provided and include, for instance, wavelengths, fluences, power levels, energy levels, temporal parameters, geometrical parameters, spot sizes, linear delivery parameters or three-dimensional delivery parameters. A spectrum of wavelengths can be selected ranging from ultraviolet to far infrared. As one skilled in the art might readily appreciate, a large number of combinations of laser beam parameters could be derived even if just two of the same lasers are used.

The present invention provides different means to select two or more laser diagnostic beams and laser beam parameters. For instance, at least one optical component could be included to adjust or control one or more laser beam parameters of one or more of laser diagnostic beams. Examples of optical component are, for instance, but not limited to, a beam profiler, a collimator, a spherical element, an a-spherical element or a parabolic element. In addition, the means to select also includes means to control each one of the lasers. Each laser can be controlled separately or by an overarching single control panel. The present invention also includes means to control one or more laser beam parameters of at least one of the laser diagnostic beams.

The present invention further includes means to deliver the laser diagnostic beams in a combined diagnostic beam. Subsequently, the combined diagnostic beam is delivered at a substance at which the substance undergoes fluorescence emission. Diagnosis in the present invention is then defined as a combination of two or more different laser diagnostic beams applied simultaneously. The type of diagnosis is dependent on the substance and the potential structural deformation(s), irregularity or irregularities, disease(s), complaint(s) or symptom(s) of the substance that one is trying to identify or diagnose. The substance in the present invention is, for instance, but not limited to, a biological tissue, a (bio)chemical compound, a bioengineering composition, a fluid, a food product or a physical structure. An example of a diagnosis is a medical diagnosis and the laser diagnostic beams in the combined diagnostic beam are medically useful diagnostic beams.

The means to deliver could include a mirror-based optical delivery device to control the combined diagnostic beam. The mirror-based optical delivery device could include linear delivery means and/or three-dimensional delivery means. The means to deliver could also include a micromanipulator, endoscopic delivery means or an optical device.

The present invention further includes means to detect the fluorescence emission. Once the fluorescence emission is detected, various different diagnostic maps and/or parameters could be analyzed or computed. Any of these diagnostic maps and/or parameters could then be displayed with various kinds of means to display to a user. The means to detect the fluorescence emission could include a plurality of optical paths such as optical fibers, articulated arms or waveguides. One or more of these optical paths could be positioned to detect a substantially uniform distribution of the fluorescence emission from the substance. Once a diagnosis is analyzed or computed, a treatment could be recommended and applied. The present invention further includes means for treating a substance.

The apparatus of the present invention could be a handheld delivery apparatus. The handheld delivery apparatus is then a portable and transferable miniature handheld delivery apparatus with, for instance, dimensions of 6″ by 12″ by 20″ or less. Such a handheld apparatus operates on an independent power source such as battery power.

In general, the method of the present invention for simultaneously delivering a combined laser diagnostic beam includes the step of selecting two or more laser diagnostic beams with each laser diagnostic beam having at least one different laser beam parameter. The method further includes the step of simultaneously delivering the laser diagnostic beams in a combined laser diagnostic beam to a substance at which the substance undergoes fluorescence emission.

The present invention also includes a computer program to control and manage the simultaneous delivery of multiple laser diagnostic beams to a substance. The computer program also includes communication means to communicate information between the computer program and one or more remote stations or users. Furthermore, the present invention includes a database that contains of a plurality of laser diagnostic plans.

In view of that which is stated above, it is the objective of the present invention to provide an apparatus and method that is able to deliver a combined diagnostic beam to a substance with the greatest variety according to the need of a diagnosis of a substance.

It is another objective of the present invention to provide a multiple laser diagnostic apparatus and method to simultaneously deliver two or more laser diagnostic beams as a combined laser diagnostic beam.

It is yet another objective of the present invention to provide a multiple laser diagnostic apparatus and method wherein each laser diagnostic beam in the combined laser diagnostic beam has at least one distinct laser beam parameter.

It is still another objective of the present invention to provide a multiple laser diagnostic apparatus and method wherein each laser diagnostic beam in the combined laser diagnostic beam has a distinct wavelength ranging from ultraviolet to far infrared.

It is still another objective of the present invention to provide means to select two or more laser diagnostic beams and laser beam parameters.

It is still another objective of the present invention to provide optical components to alter or control one or more laser beam parameters of one or more laser diagnostic beams in the combined laser diagnostic beam.

It is still another objective of the present invention to provide control of two or more lasers.

It is still another objective of the present invention to provide control of one or more laser beam parameters.

It is still another objective of the present invention to preserve the mode of each laser diagnostic beam.

It is still another objective of the present invention to provide a mirror-based delivery device to control the combined laser diagnostic beam.

It is still another objective of the present invention to provide a multiple laser diagnostic apparatus and method with linear scanning and delivery capability of the combined laser diagnostic beam.

It is still another objective of the present invention to provide a multiple laser diagnostic apparatus and method with three-dimensional scanning and delivery capability of the combined laser diagnostic beam.

It is still another objective of the present invention to detect the fluorescence emission.

It is still another objective of the present invention to compute or analyze various different diagnostic maps and/or parameters.

It is still another objective of the present invention to display diagnostic data to a user.

It is still another objective of the present invention to provide means to detect a substantially uniform distribution of the fluorescence emission from the substance.

It is still another objective of the present invention to provide a computer program to control and manage the simultaneously delivery of multiple laser diagnostic beams to a substance with a multiple laser diagnostic apparatus and method.

It is still another objective of the present invention to provide a database of diagnostic plans for laser applications wherein two or more laser diagnostic beams are simultaneously delivered to a substance.

It is still another objective of the present invention to provide means for treating the substance based on the diagnosis.

The advantage of the present invention over the prior art is that the apparatus enables one to perform a diagnostic plan with the greatest variety of laser diagnostic beams at the same time. Another advantage of the present invention is that it enables one to deliver two or more different laser diagnostic beams simultaneously in a combined beam to a substance wherein each laser diagnostic beam has at least one different laser beam parameter. Yet another advantage of the present invention is that it significantly decreases the overall laser diagnostic and operation time. Still another advantage of the present invention is that it provides for the means to advance laser diagnostic plans or recipes with combined laser diagnostic beams for simultaneous delivery to a substance.

### BRIEF DESCRIPTION OF THE FIGURES

The objectives and advantages of the present invention will be understood by reading the following detailed description in conjunction with the drawings, in which:

FIG. 1 shows an example of a multiple laser diagnosis apparatus and method according to the present invention;

FIG. 2 shows an example of a multiple laser diagnostic apparatus and method wherein optical components are included to select the laser beam parameters according to the present invention;

FIG. 3 shows an example of a multiple laser diagnostic apparatus and method with means to control according to the present invention;

FIGS. 4-7 shows different examples of two different laser diagnostic beams in a combined beam according to the present invention;

FIG. 8 shows an optical device to select and combine laser diagnostic beams according to the present invention;

FIG. 9 shows a mirror-based delivery means;

FIG. 10 shows another example of a multiple laser diagnosis apparatus and method according to the present invention;

FIG. 11 shows an example of a detection means according to the present invention;

FIG. 12 shows an example of a multiple laser diagnosis apparatus and method including a treatment means according to the present invention;

FIG. 13 shows a flow diagram of a computer program according to the present invention;

FIG. 14 shows an illustration of a communication system between the apparatus and method of the present invention and remote agents.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

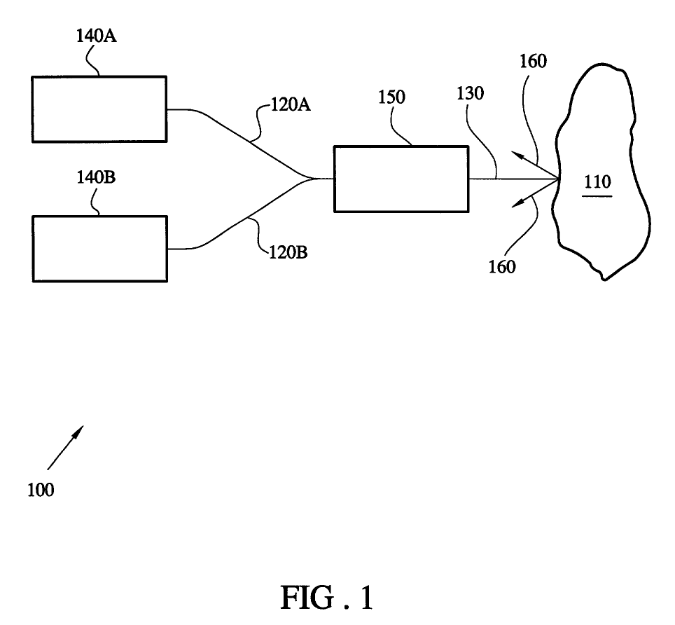

The present invention provides a multiple laser diagnostic apparatus and method 100, as shown by an exemplary embodiment in FIG. 1, that provides versatility and flexibility in diagnosing a substance 110 with multiple laser diagnostic beams 120A and 120B at the same time. In general, the present invention provides an apparatus and method wherein two or more laser diagnostic beams, with each laser diagnostic beam having at least one distinct laser beam parameter, are selected and delivered simultaneously in a combined laser diagnostic beam 130 to substance 110 with the purpose to fluoresce 160 substance 110. Combined laser diagnostic beam 130 is also referred to as combined beam 130. The delivery of combined beam 130 enables one to perform two or more different diagnosis at the same time to substance 110 instead of just one single diagnosis or single wavelength each time as is most common in the prior art. Diagnosis in the present invention is then defined as a combination of two or more different laser diagnostic beams applied simultaneously. Diagnosis is also refers to any type of fluorescence-induced diagnosis. The type of diagnosis is dependent on substance 110 and the potential structural deformation(s), irregularity or irregularities, disease(s), complaint(s) or symptom(s) of substance 110 that one is trying to identify or diagnose. In general, physical, chemical, biological or pathological condition of substance 110 could be determined. Substance 110 could be any type of substance, but is preferably a substance with different compositions or structures such as, but not limited to, biological tissue, (bio)chemical compounds, bioengineering compositions and physical structures or materials. In case of biological tissue, combined beam 130 is, for instance, applied in surgical or endoscopic surgery wherein different cells or tissue are diagnosed with different laser diagnostic beams 120A, 120B to 120C and simultaneously delivered to substance 110 by combined beam 130. Examples of surgical or endoscopic surgery are, for instance, but not limited to, dermatological, urological (prostate), myringotomy, cardiovascular, neurological, otolaryngological, or visual procedures. One could diagnose, for instance, but not limited to, blood flow parameters, blood disorders, cancerous tissue, dead or alive tissue, tissue composition, artificial implants, survivability of tissue or proteins. In case of (bio)chemical compounds, combined beam 130 is, for instance, applied in tissue or genetic engineering wherein different laser diagnostic beams 120A and 120B could, for instance, but not limited to, diagnose different parts of DNA or quality of chemical compositions as they are simultaneously delivered to substance 110 by combined beam 130. In case of materials, combined beam 130 is, for instance, applied in material engineering or semiconductor applications, wherein different laser diagnostic beams 120A and 120B simultaneously diagnose various parts of the structure as these parts convey different types of fluorescence emission 160. As one skilled in the art might readily appreciate, various different examples could be developed and the present invention is not limited to the above mentioned examples. The present invention could also include the diagnosis of food products or fluids in which cases contamination of a food or fluid could be detected or identified (see, for instance, an article published by Dale Ott entitled “Fluorescent proteins reveal food contamination” published in BioPhotonics International, November 2001 page 18-19).

The example shown in FIG. 1 includes two lasers 140A and 140B, however, the present invention generally includes two or more lasers. Each laser simultaneously delivers a laser diagnostic beam. Each laser diagnostic beam has at least one distinct laser beam parameter. The lasers can be different lasers or the same type of lasers. In case of the same type of laser at least one laser beam parameter in each laser diagnostic beam is different. In general, one or more laser beam parameters of the laser diagnostic beams a re different. However, one or more laser beam parameters of the laser diagnostic beams can also be the identical. Several different types of lasers could be employed, such as, but not limited to, different type gas lasers (such as CO₂, excimer, argon, cu-vapor lasers), flashlight laser, liquid lasers (dye lasers) or solid state lasers (such as YAG, semiconductor, Ti: sapphire lasers). The present invention is not limited to a pulsed laser or a continuous wave laser. Coherent Inc. provides a product line with a wide variety of diode lasers that each have a different wavelength or wavelength range. For instance, Coherent's product line encompasses continuous wave (CW) laser diode bars, single stripe CW, conduction cooled quasi continuous wave (QCW) laser diode bars, fiber array packaged bars, or all kinds of integrated packages. In addition, Coherent's product line of Sapphire lasers (e.g. the solid state 488 nm laser) could be used.

Each laser can be controlled or programmed to select and deliver different laser diagnostic beams 120A and 120B simultaneously. The different laser diagnostic beams are combined by delivery means 150 into combined beam 130. Combined beam 130 is delivered at substance 110. Each laser diagnostic beam 120A and 120B could be transmitted to and from delivery means 150 by any type of suitable optical path. Examples of optical paths that could be used are, for instance, but not limited to, an optical fiber, an articulated arm or a waveguide. As is described in detail below, delivery means 150 could, for instance, also include an optical device, a micromanipulator or a mirror-based optical delivery device. Combined beam 130 could either be directly delivered by delivery means 150 to substance 110 or could be further transmitted by, for instance, an optical fiber or a waveguide inside substance 110 as is, for instance, but not limited to, useful in endoscopic procedures.

Laser beam parameters are, for instance, but not limited to, wavelengths ranging from ultraviolet to far infrared, fluences, power levels, energy levels, temporal parameters, geometrical parameters, spot size, linear delivery parameters or three-dimensional delivery parameters. As one skilled in the art might readily appreciate, the present invention provides a platform to advance diagnostic plans or recipes with a combination of two or more laser diagnostic beams for simultaneous delivery to substance 110 and diagnosis of substance 110. An example, which is illustrative rather than restrictive, of some laser beam parameters as they are known in the prior art is for the diagnostics of food contamination. It is known that different fluorescent proteins are excited by different wavelengths of light: cyan by 477 nm, green by 488 nm, yellow by 527 nm, red by 558 nm. The teaching of the present invention can combine two or more of these wavelengths in a combined beam. As one skilled in the art might readily appreciate, a large number of combinations of laser beam parameters could be derived even if just two of the same lasers are used. An example is, for instance, that two of the same lasers are used each delivering a laser diagnostic beam with the same wavelength, however, each laser diagnostic beam is delivered at a different power level; e.g. laser 1 could use only 10% of the power and laser 2 could only use 90% of the power. Another example, is that two of the same lasers are used each delivering a laser diagnostic beam with the same wavelength, however, each laser diagnostic beam is delivered with different geometrical beam parameters. Geometrical beam parameters are, for instance, but not limited to, the diameter of a beam, the focus point of a beam or the de-foci footprint(s) of a beam.

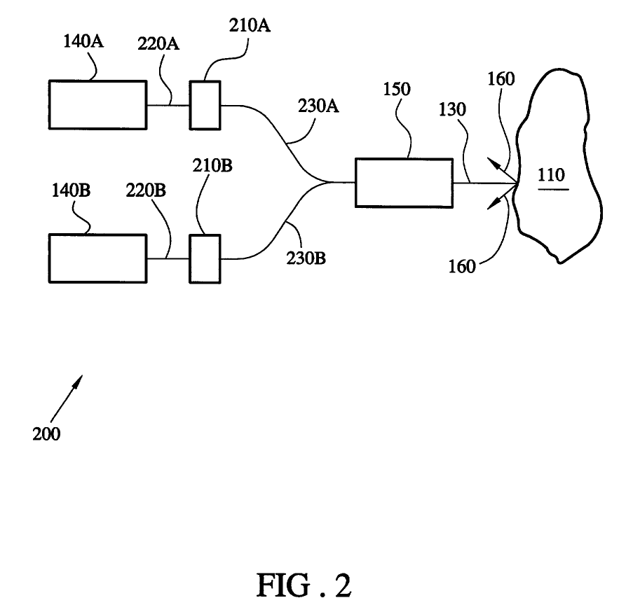

There are several different ways to select, by adjusting or controlling, the laser beam parameters for a particular diagnosis. For example, laser beam parameters can be selected by adding hardware components, such as one or more optical elements, to apparatus and method 100 to change a laser beam parameter. FIG. 2 shows an exemplary embodiment of a multiple laser diagnostic apparatus and method 200 that is similar to FIG. 1 with the addition of optical components 210A and 210B that could select by adjusting or controlling one or more beam parameters of laser diagnostic beams 220A and 220B into selected laser diagnostic beams 230A and 230B respectively. FIG. 2 shows one optical component for each laser diagnostic beam, but there is no limitation to the number of optical components that could be used to select a laser beam parameter. Examples of optical components 210A and 210B include, for instance, but are not limited to, a collimator, a spherical element, an a-spherical element, a parabolic element, or any other optical element that could select the beam parameter of the laser diagnostic beam.

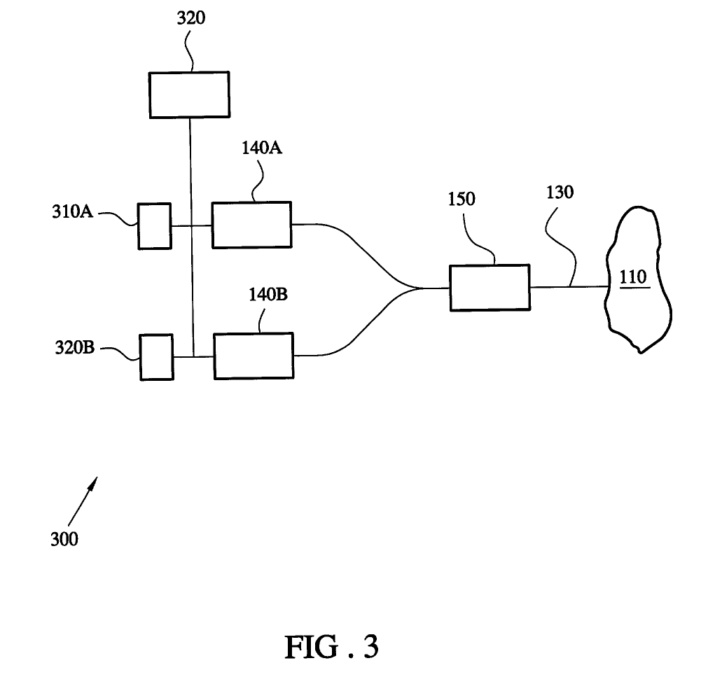

Another way to select laser beam parameters is by a control means that allows one to control, for instance, one or more lasers or one or more laser beam parameters. FIG. 3 shows an exemplary embodiment of a multiple laser diagnostic apparatus and method 300 that is similar to FIG. 1 with the addition of control means 310A and 310B that are linked directly to laser 140A and 140B respectively, or control means 320 that is a single control panel that is linked to all lasers 140A and 140B. Examples of control means include, for instance, but are not limited to, a software panel or interface with virtual control panels and buttons or a hardware panel with control buttons. Control means 310A and 310B or control means 320 enables a user to control, for instance, but not limited to, the selection of wavelengths, the energy (or fluence) of each wavelength, the intensity (or power), the temporal parameters (such as pulse parameters and repetition rate) of each laser diagnostic beam and the repetition rate of the combination of wavelengths or each individual wavelength. As one skilled in the art might readily appreciate, these laser beam parameters affect the fluorescent effect of its diagnostic laser beam.

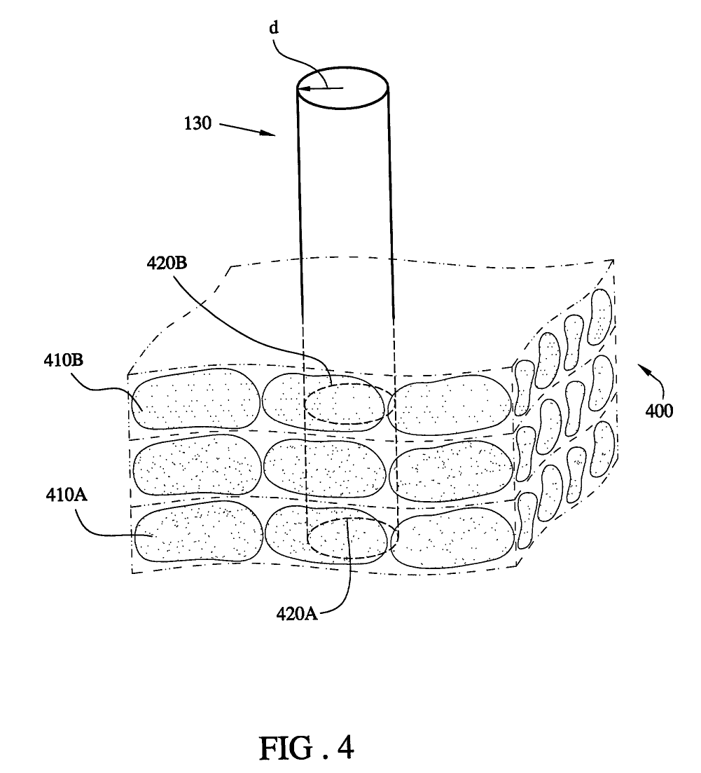

As mentioned above, a large number of different laser diagnostic beams could be combined. FIGS. 4-7 show some illustrative examples of two different laser diagnostic beams in combined beam 130. FIG. 4 shows an example of substance 400 that could, for instance, be a biological tissue with different cells 410A and 410B. The diagnostic plan may require a combined beam 130 that includes two laser diagnostic beams 420A and 420B. The wavelength and tissue penetration depth are different for laser diagnostic beam 420A and 420B. For instance, laser diagnostic beam 420A targeting cells 410A is delivered by a HeNe laser with a wavelength of 633 nm and laser diagnostic beam 420B targeting cells 410B is delivered by a Er:YAG laser with a wavelength of 2940 nm. Laser diagnostic beams 420A and 420B in combined treatment beam 130 have similar geometrical parameters as shown by diameter d of laser diagnostic beams 420A and 420B.

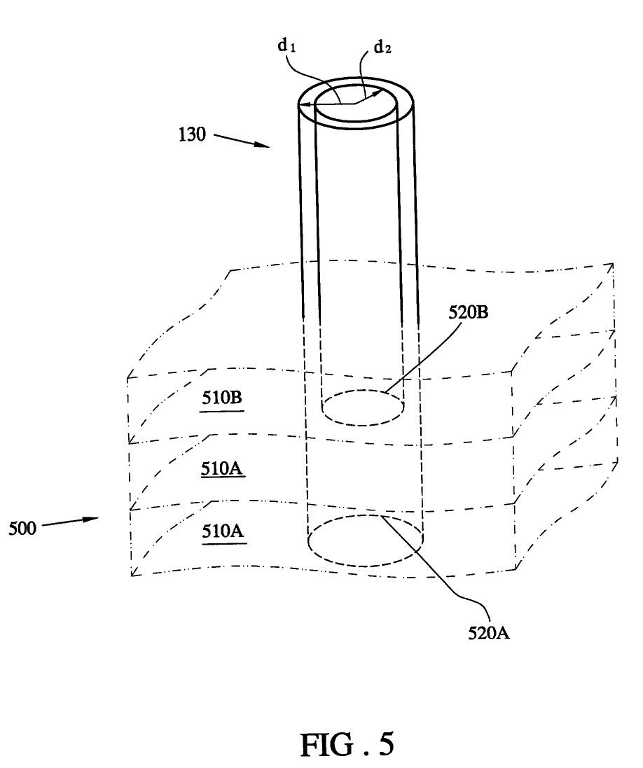

FIG. 5 shows an example of substance 500 that could, for instance, be a biological tissue with different tissue layers 510A and 510B. The diagnostic plan may require a combined beam 130 that includes two laser diagnostic beams 520A and 520B. In this example, the wavelength, penetration depth as well as laser beam diameter are different for laser diagnostic beams 520A and 520B. For instance, laser diagnostic beam 520A targeting layer 510A is delivered by a CO₂ laser with a wavelength of 10,600 nm and laser diagnostic beam 520B targeting layer 510B is delivered by a Alexandrite laser with a wavelength of 755 nm. Laser diagnostic beam 520A and 520B in combined beam 130 have different geometrical parameters as shown by diameter d₁ of beam 520A and diameter d₂ of beam 520B.

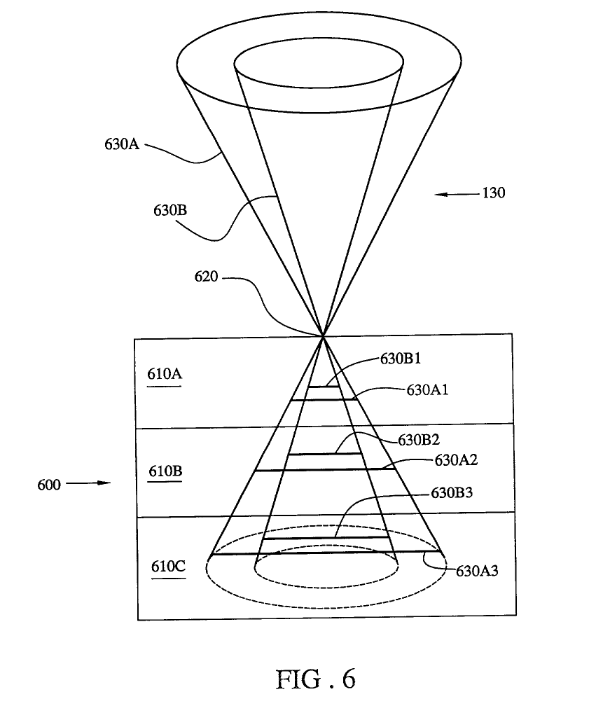

FIG. 6 shows an example of substance 600 that could, for instance, be a physical structure with different materials 610A, 610B and 610C. The diagnostic plan may require a similar focus point 620 of laser diagnostic beams 630A and 630B that are combined in combined beam 130. However, the key aspect of this particular diagnosis might be to have establish different fluorescent effects at different de-foci footprints 630A1, 630A2 and 630A3 for laser diagnostic beam 630A and 630B1, 630B2 and 630B3 for laser diagnostic beam 630B.

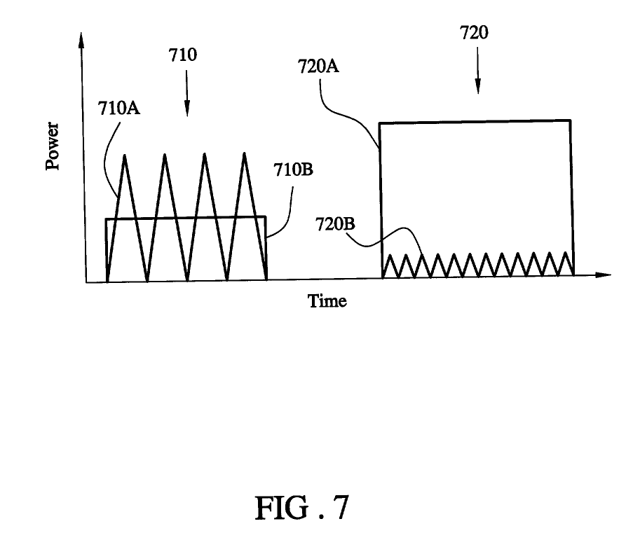

FIG. 7 shows an example of two combined laser diagnostic beams 710 and 720 wherein the laser diagnostic beams have different temporal parameters. Temporal parameters of a laser diagnostic beam are, for instance, but not limited to, the pulse repetition rate, duration of the pulse and overall radiation time of the laser diagnostic beam. For example, combined diagnostic beam 710 has a high repetition, high power beam 710A and a low power, continuous beam 710B. In the other example, combined diagnostic beam 720 has a long pulse, high power beam 720A and a short pulse, low power beam 720B.

As mentioned above, delivery means 150 could, for instance, include a micromanipulator (e.g. micromanipulator 710/711 Acuspot by Sharplan Lasers Inc., micromanipulator by TTI Medical Inc. or Cryomedics micromanipulator by Cabot Medical Inc.), an optical device or a mirror-based optical delivery device. The preferred delivery means 150 is a device that preserves the mode of each laser diagnostic beam.

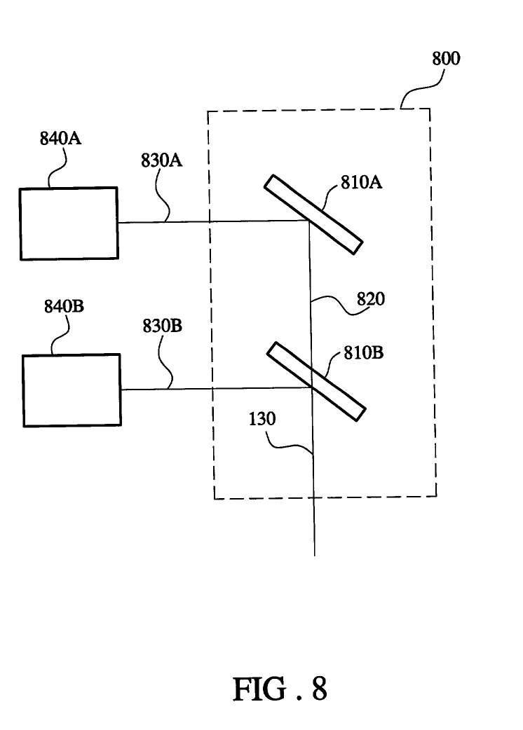

FIG. 8 shows an exemplary embodiment of delivery means 150 that includes an optical device 800. Device 800 includes optical components 810A and 810B that are aligned on an optical path 820 to receive laser diagnostic beams 830A and 830B from lasers 840A and 840B respectively. Each optical component 810A and 810B directs and selectively combines laser diagnostic beams 830A and 830B along optical path 820. Examples of the various kinds of optical components that can be used are, for instance, a wavelength selective mirror, a wavelength selective filter, a beam splitter, or any other optical device that is capable of directing and selectively combining different laser diagnostic beams that are selected to create combination 130. An illustrative example of such a mirror is, without being restrictive, a Silflex MK-II mirror by Unaxis-Balzers Inc. This mirror has high reflectivity values through the visual, near, middle and far infra red. Optical device 800 could further include position or rotation means (not shown) to control the linear position or angular position of optical components 810A and 810B with respect to optical path 820. Position or rotation means could be established by various different techniques such as, for instance, an optical switching device, a folding beam splitter, a piezo-electric element, a solenoid, a preprogrammed stepper motor, or the like. Positioning of optical components 810A and 810B is, for instance, related to removing an optical component away from the optical path if the optical component was already positioned in the optical path. A reason for removing an optical component is, for instance, based on a selection by a user that the particular laser diagnostic beam outputted by the corresponding laser is no longer necessary in the selected combination or possibly interferes with the selected combination. Rotating optical components 810A and 810B is, for instance, related to redirect one or more laser diagnostic beams to generate a subset of combinations of the laser diagnostic beams. Position or rotation means is also meant for aligning or realigning optical components 810A and 810B along optical path 820.

Since the present invention involves a combination of laser diagnostic beams each having different laser beam parameters, a lens based system to deliver combined beam 130 would not only be impractical, but would also cause chromatic aberration. In addition, a lens based system cannot be focused to a spot size smaller than 0.4 mm. Therefore in order to more practically and more accurately focus combined beam 130 on a desired spot, it is necessary that delivery means 150 includes a mirror-based optical delivery device to control the focus of combined beam 130 on substance 110. U.S. Pat. Nos. 5,955,265 and 5,163,936 (both hereby incorporated by reference) assigned to the same assignee as the present invention, discloses a mirror-based optical delivery device that was invented to avoid chromatic aberration and better focus a laser beam by aligning a visual beam with the laser beam wherein the visual beam is solely used to visually guide the laser beam. The mirror-based optical delivery device is preferred as delivery means 150 in the present invention to more practically and more accurately focus combined beam 130 at substance 110. In the present, invention mirror-based optical delivery device delivers and controls two or more different laser diagnostic beams to substance 110.

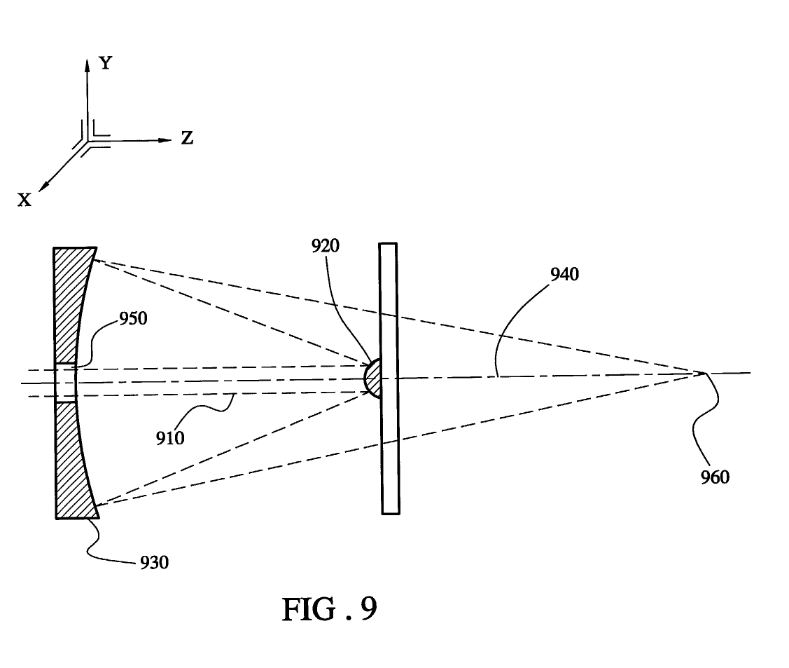

U.S. Pat. No. 5,128,509 (hereby incorporated by reference) to the present inventor and assigned to the same assignee as the present invention discloses a mirror-based optical delivery device 900 as shown in FIG. 9, which uses reflective optics to steer and focus combined beam 910. The optical focusing of device 900 is performed by a convex mirror 920 and a concave mirror 930 facing each other and aligned on a common optical axis 940. Combined beam 910 passes through a small hole 950 in the center of concave mirror 930 and is reflected by convex mirror 920 back towards concave mirror 930. Concave mirror 930 reflects the beam forward to a focus 960 beyond convex mirror 920. Because this device uses reflective optics, it is capable of delivering laser diagnostic beams of a wide range of wavelengths and laser beam parameters and to a very small focus. With the mirror-based optical delivery device, the present invention enables one to deliver combined beam on substance with a spot size that is 0.1 mm or less. Alternatively, the present invention is not restricted to allow one to deliver combined diagnostic beam on substance with a spot size that is 0.1 mm or more. Unlike systems using refractive optics, mirror-based optical delivery device 900 enables one to simultaneously deliver coincident laser diagnostic beams ranging from ultraviolet to far infrared. Moreover, because reflective optics do not exhibit chromatic aberration, mirror-based optical delivery device 900 delivers combined beam 910 with two or more laser diagnostic beams to the same focal point.

Mirror-based optical delivery device 900, however, does not provide a means for scanning to produce a uniform exposure over a large surface area. U.S. Pat. No. 5,995,265 to the present inventor and assigned to the same assignee as the present invention discloses a mirror-based optical delivery device with linear scanning or delivery means to scan a diagnostic area with a predetermined linear scanning or delivery pattern. To establish a linear scanning or delivery pattern different control means (not shown) are included to rotate concave mirror 930 and/or convex mirror 920 around the X, Y and/or Z-axis. In the present invention, laser diagnostic beams delivered in combined diagnostic beam could generate various different kinds of diagnostic patterns, such as a spiral diagnostic pattern to cover an elliptical region rather than a circular one. In addition, the diagnostic pattern can be adjusted to cover annular regions and elliptically annular regions. The diagnostic pattern can also be adjusted so that the combined beam follows a circular or elliptical path rather than a spiral path. The path can also be adjusted to follow other types of paths, such as a Lissajous figure. Of course, by fixing the mirrors, combined beam may be directed to a single point as well. Since the path of combined beam could be controlled by a microprocessor programming device or by hand, the types of paths and diagnostic patterns are not limited to any single class.

U.S. Pat. No. 5,995,265, however, does not teach a means for three-dimensional scanning to produce a depth exposure over a large area. The present invention further includes a mirror-based optical delivery device with three-dimensional scanning or delivery means to diagnose a three-dimensional area with a three-dimensional scanning or delivery pattern. An example of how a three-dimensional scanning or delivery pattern could be established is, for instance, by combining linear scanning means as described above with control means (such as one or more stepper motors, not shown) that is capable of changing the relative position of convex mirror 920 and a concave mirror 930 along common optical axis 940, i.e. to translate concave mirror 930 and convex mirror 920 over the Z-axis with respect to each other. As one skilled in the art might readily appreciate, several different control means could be developed to control the relative position between convex mirror 920 and a concave mirror 930 along common optical axis 940. The path of combined beam 910 could be controlled by a microprocessor programming device or by hand which enables one to create any type of three-dimensional path and diagnostic pattern. The delivery means of the present invention allows one to deliver a diagnostic pattern in a static manner or in a dynamic manner where the three-dimensional diagnostic patterns changes shape and location at the substance during the diagnosis.

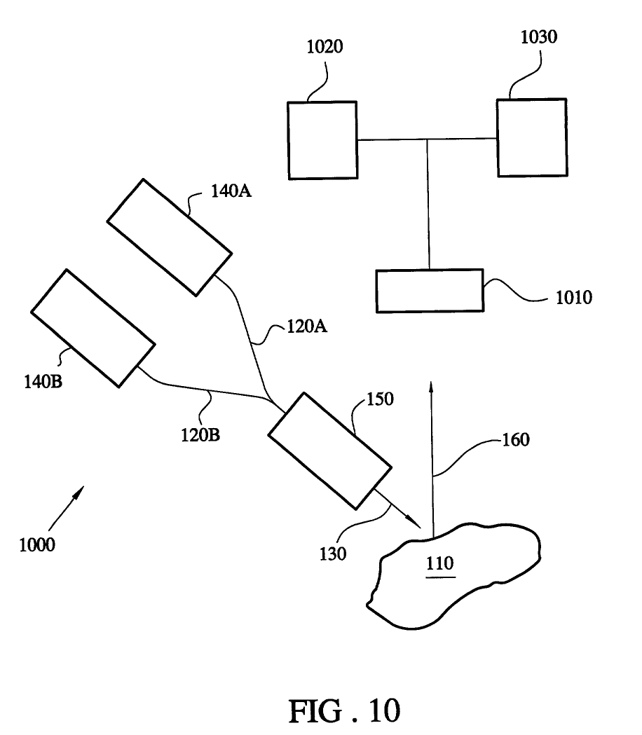

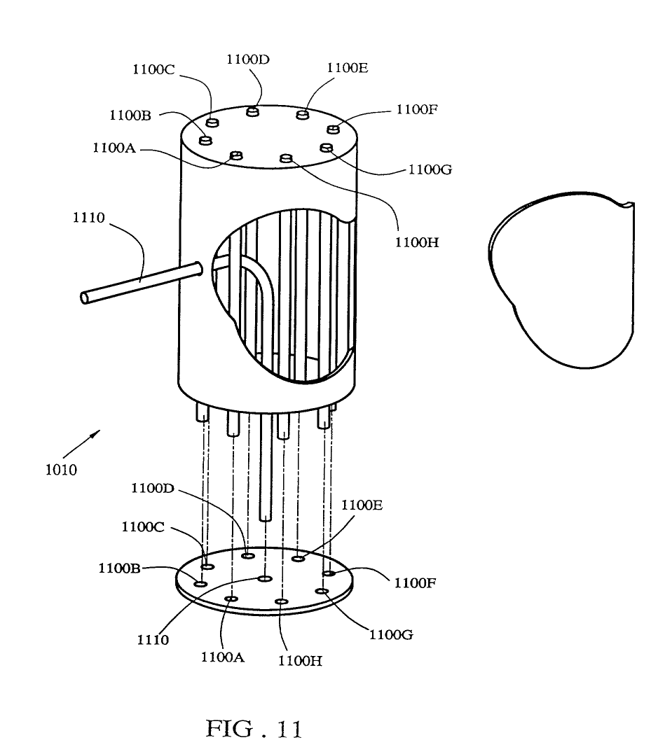

The fluorescence emission 160 is detected by a detection means 1010 as shown in multiple laser diagnostic apparatus and method 1000 in FIG. 10. Detection means 1010 can be any type of detection device capable of detecting fluorescence emission 160. In a preferred embodiment as shown in FIG. 11, detection means 1010 includes a plurality of optical paths 1100-A . . . 1100-H. The plurality of optical paths 1100-A . . . 1100-H are used to detect fluorescent emission 160 (also called detection, collection or reader paths) and are combined in a device with optical path 1110 that is used to deliver combined diagnostic beam 130 (also called excitation path). Furthermore, the delivery and detection optical paths could be optical fibers, however, they could also be other types of optical paths such as articulated arms or waveguides. In another preferred embodiment of detection means 1010, detection fibers 1100-A . . . 1100-H are arranged or positioned in such a way that fluorescent emission 160 is detected with a substantially uniform distribution. The uniform distribution is related to the uniformity of the diagnosed area and/or to the uniformity of the coverage (or intensity) per area. To establish such a uniform distribution, one or more than one optical fiber in this arrangement is positioned or cleaved, for instance, under angle to make sure that the area under diagnosis is covered substantially in its entirety. An increased uniformity increases the accuracy of a diagnosis. When the accuracy of a diagnosis increases it is self-evident that the accuracy of the diagnosis and potentially a subsequent treatment becomes more accurate. This is of importance when one, for instance, follows the progression of cancerous tissue, determines the reliability of artificial components or structures, or in any kind of endoscopic procedure or treatment.

FIG. 10 further shows computing or analyzing means 1020 that computes and/or analyzes various data from the detected fluorescence emission by detection means 1010. Computing or analyzing means 1020 is preferably a computing device such as a computer. However, computing or analyzing means 1020 could be any device that is capable of analyzing the detected fluorescence emission, compute graphs or maps and/or determine parameters that could be used for the diagnosis. Other information that could be useful for analysis or computation is, for instance, but not limited to, a check for (in)consistency, a check for a change in spread as might be interesting in diagnosing cancerous tissue, a change in color or intensity of the emission spectra. In general, any type of quantity or quality data of a tissue, composition, structure or product could be analyzed or computed.

Computing or analyzing means 1020 also include means to create a map based on the detected fluorescence emission. Various different means to create a map, such as computer programs or routines, are available in the art and could be included. A two-dimensional or three-dimensional map of the diagnosed area could be created. Each map could be an overview map of the entire area or a close-up view of a particular area of interest. Maps could be stored in a database and accessed by a computer program. Computing or analyzing means 1020 further includes means to interpret and/or analyze the fluorescence maps in terms of a pattern or map. Once the pattern or map has been analyzed, a computer program could be further employed to diagnose a particular complaint, disease or deformation of substance. Computer program could access a database of patterns to allow for a comparison and/or analysis of the detected pattern with one or more patterns in the database.

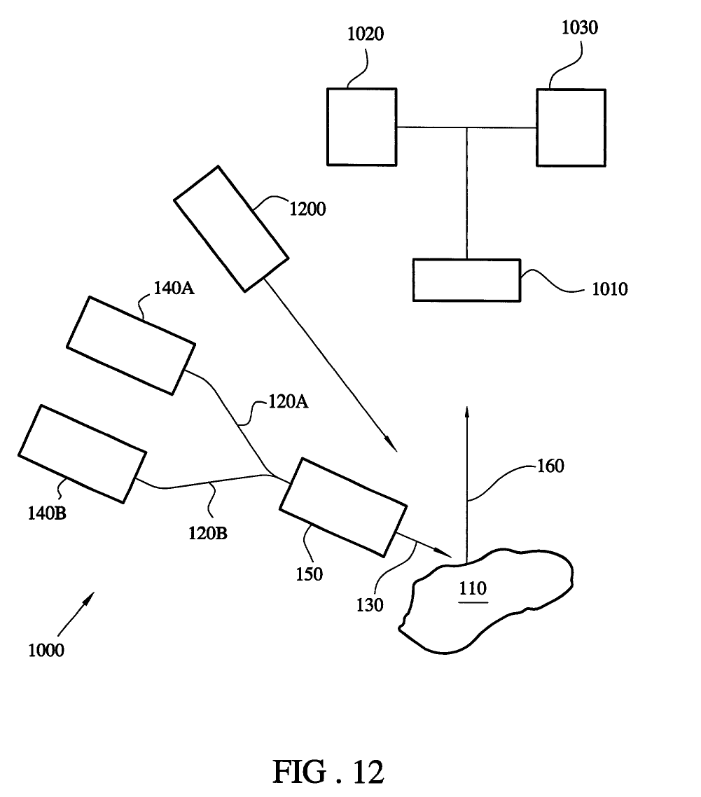

Once an analysis or computation has been performed, computing or analyzing means 1020 could indicate a type of diagnosis. The indication could be based on a comparison of the analyzed fluorescence emission with knowledge that previously obtained. Such a comparison or analysis could either be done automatically by, for instance, pixel comparison or manually where a graphical user interface enables the user to perform such an comparison or analysis. Computing or analyzing means 1020 further includes means to allow to also recommend and/or (automatically) execute a treatment protocol. Computing or analyzing means 1020 could recommend any type of treatment. Such a treatment could, for instance, be a laser treatment, a photodynamic therapy or pharmaceutical treatment. FIG. 12 shows an example of a multiple laser diagnostic apparatus and method 1000 combined with a treatment means 1200. Treatment means 1200 could either be a separate module or an integral part of the multiple laser diagnostic apparatus and method of the present invention. Treatment means includes a treatment system that enables a user to apply a laser treatment that, for instance, was recommended by analyzing or computing means as described above. The present invention could include any type of treatment means 1200. A preferred treatment means 1200 is a multiple laser treatment apparatus and method wherein two or more laser treatment beams are used and applied simultaneously in a combined treatment beam to treat a substance. Details regarding such a multiple laser treatment apparatus and method is disclosed in copending US patent application entitled “Multiple Laser Treatment” by the same inventor as the present invention and having the same filing date as the present invention. This copending application is incorporated by reference for all that is discloses.

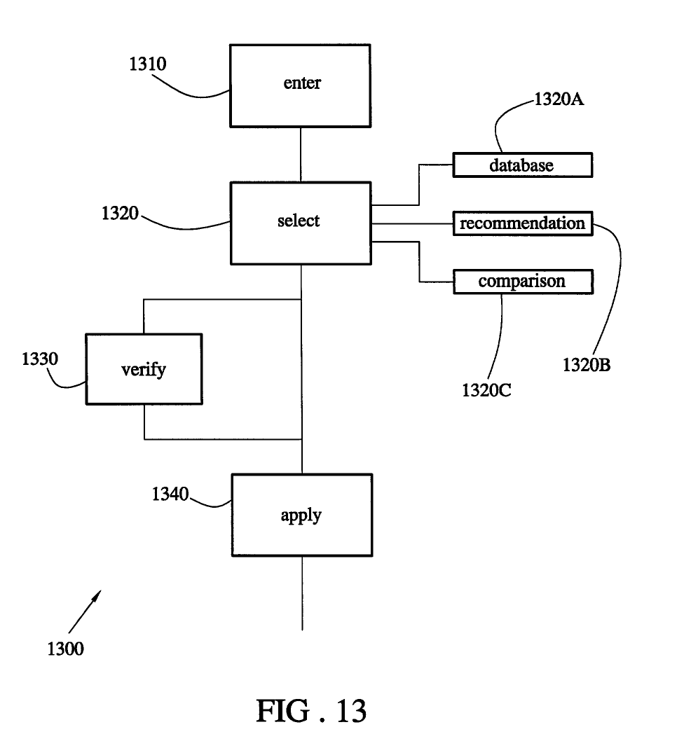

Computing or analyzing means 1020 can be implemented by a variety of computer programs or means such as C⁺⁺, Java, Unix, HTML, XML and the like. Computing or analyzing means 1020 can also be implemented on different hardware devices, such as computer devices, handheld devices and the like. Computing or analyzing means 1020 could further include a computer program 1300 as shown in FIG. 13. Computer program 1300 could manage and control the simultaneously delivering multiple laser diagnostic beams to a substance with a laser diagnostic system. Computer program 1300 provides means to enter data 1310. Means to enter data are, for instance, but not limited to, a keyboard, a touch-screen, a handheld device, a web-based application, a voice recognition system and the like. Computer program 1300 is not limited to any other means for entering data. The type of data that can be entered is, for instance, but not limited to, the type of lasers, the type of laser diagnostic beams, laser beam parameters, substance information, diagnostic protocols, complaint information, disease information, etc. In the example of a patient that needs to undergo a laser diagnosis, data can also include patient information data including patient visits and type of previous or related diagnoses. Computer program 1300 also provides means for selecting a diagnostic plan 1320. Means for selecting 1320 are, for instance, but not limited to, through a keyboard, a touch-screen, a handheld device, a web-based interaction, a voice recognition system and the like. Computer program 1300 can select a diagnostic plan from a database 1320A that contains, for instance, predetermined diagnostic plans. A diagnostic plan can also be selected based on a recommendation 1320B of a diagnostic plan which is based on, for instance, previous diagnosis trials or intelligent reasoning, or comparison 1320C based on entered data 1310. Guidance or recommendation is established by having knowledge stored in a database that can be accessed or requested from the computer program. The computer program could then respond by providing a list of choices and recommendations after which the user could either select or modify the provided choices and subsequently perform the procedure. Once the diagnostic plan has been established, the user has the opportunity to verify 1330 the selected diagnostic plan before it is applied 1340 to the substance. Different means for verifying 1330 the diagnostic plan could be used such as, for instance, but not limited to, visual inspection of the list of laser beam parameters, including boundaries and/or warnings for the laser beams parameters or combination of laser diagnostic beams, statistical verifications or calculations and the like. The verifying means 1330 is not limited to verifying the combined diagnostic beam before it is applied to the substance since it can also be verified in a simulation or virtual environment. The user could also verify the combined beam by actually applying combined beam at a test substance. The user could also elect to have verifying means as an optional step in computer program 1300. This optional step makes most sense if the diagnosis is a standard approach and used on a routine basis. Means to apply 1340 the combined diagnostic beam encompasses any software or hardware connection that allows the program to control the multiple laser diagnostic apparatus. These type of connections are well known in the art.

Useful information, related to laser diagnostic plans wherein two or more laser diagnostic beams are delivered simultaneously, could be stored in a database. The database could, for instance, be accessed by computer program 1300. Such a database provides information of a plurality of diagnostic plans that specifies the type of lasers and laser beams parameters. As one skilled in the art would readily appreciate, such a database could include various kinds of related parameters such as substance-related information, patient-related information, etc. In general the type of data in the database depends on the type of diagnostic plan which varies from any type of medical diagnostic plan, any type of (bio)chemical or bioengineering diagnostic plan or any type of physical diagnostic plan.

Referring back to FIG. 10, a displaying means 1030 is shown to display the detected fluorescence emission and/or diagnosis by detection means 1010. Displaying means 1030 could be any type of displaying device such as, for instance, but not limited to, a computer monitor, handheld computer device, flat panel display, CCD or the like. As indicated in FIG. 10, displaying means 1030 could also display any of the data of computer program 1100 and all kinds of computed graphs, maps and/or parameters that are useful for the diagnosis. In addition, displaying means 1030 could also show combinations of diagnosed data with any other type of related data, graphs or maps such as CadScans, MRIs, X-rays and the like.

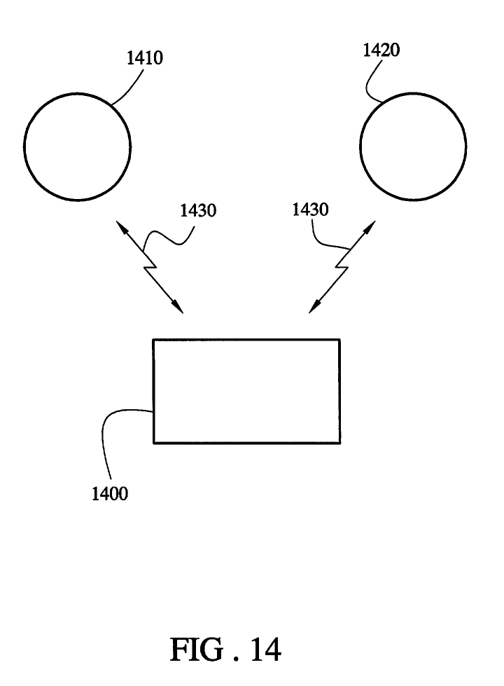

Computer program 1100 also includes different ways of communicating 1430 data or information as shown in FIG. 14 between a user or an another computer, indicated by remote station 1410 and 1420. Remote station 1410 and 1420 could, for instance, contain a useful database, new information for diagnostic plans, or any other useful information for any laser diagnostic plan. Other information that could be exchanged is mailing list information or software updates. Means of communicating are, for instance, but not limited to, wireless communication means or any type of conventional communication means to communicate data as they are known in the art.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For instance, the apparatus of the present invention could easily be developed as a handheld delivery apparatus. This handheld delivery apparatus is preferably portable and transferable to enable one to use the apparatus at various different places and circumstances. A preferred embodiment of handheld delivery apparatus is a miniature handheld delivery apparatus with dimensions of 6″ by 12″ by 20″ or less. Furthermore, the handheld delivery apparatus could be fully operational by independent power such as battery power. In addition, many different optical components can be used to select or establish the desired combination of laser diagnostic beams. The present invention could also include different means as part of the delivery means to preserve the mode of each laser diagnostic beam. In addition, the present invention includes means to vary or continuously change the pattern of the laser beams during the performance of a diagnosis. The present invention could be used in many different applications including other (bio)medical, bioengineering and industrial applications. In addition, the present invention could be used in any type of surface diagnostics or any type of endoscopic diagnostics. A variety of computer programs, environments and user interfaces can be used to control the various hardware and software components that encompasses the present invention. In addition, various kinds of display mechanism can be used such as to head-sets and glasses (see e.g. U.S. Pat. Nos. 5,114,218, 5,151,600, 5,184,156 and 5,382,986 all assigned to the same assignee as the present invention) or flat panel devices to give the user control and feedback of the diagnostic protocol and procedure. The present also includes various means to enhance fluorescence emission effect by adding fluorescent emission enhancers to the substance under diagnosis. Examples of such fluorescence emission enhancers are, for instance, but not limited to, a metallic particle (See, for instance Intensified Fluorescence by Lakowicz et al., in Photonics October 2001, pages 97-10), a dye (See, for instance Fluorescent imaging in a glioma model in vivo by Nikas et al., in Lasers in Surgery and Medicine 29:11-17, 2001), an agent, a fluorescent probe or the like. All such variations are considered to be within the scope and spirit of the present invention as defined by the following claims and their legal equivalents.

What is claimed is:

1. A multiple laser diagnostic apparatus, comprising:
(a) n lasers, wherein said n>1 and each of said n lasers simultaneously delivers a laser diagnostic beam selected for a diagnosis using fluorescence emission wherein each one of said laser diagnostic beam comprises at least one different laser beam parameter; and
(b) means to deliver said laser diagnostic beams in a combined treatment beam wherein said combined diagnostic beam is delivered at a substance at which said substance undergoes said fluorescence emission.

2. The apparatus as set forth in claim 1, wherein said laser beam parameters are wavelengths, fluences, power levels, energy levels, temporal parameters, geometrical parameters, spot sizes, linear delivery parameters or three-dimensional delivery parameters.

3. The apparatus as set forth in claim 2, wherein said wavelength is selected from a spectrum of wavelengths ranging from ultraviolet to far infrared.

4. The apparatus as set forth in claim 2, wherein said one or more laser beam parameters of said laser diagnostic beams are different.

5. The apparatus as set forth in claim 2, wherein said one or more laser beam parameters of said laser diagnostic beams are identical.

6. The apparatus as set forth in claim 1, further comprising means to detect said fluorescence emission.

7. The apparatus as set forth in claim 1, further comprising means to analyze said detected fluorescence emission.

8. The apparatus as set forth in claim 1, further comprising means to display said diagnosis.

9. The apparatus as set forth in claim 1, wherein said means to deliver comprises means to detect said fluorescence emission.

10. The apparatus as set forth in claim 9, wherein said means to detect comprises a plurality of optical paths.

11. The apparatus as set forth in claim 10, wherein said optical paths are optical fibers, articulated arms or waveguides.

12. The apparatus as set forth in claim 10, wherein one or more of said plurality of optical paths are positioned to detect a substantially uniform distribution of said fluorescence emission from said substance.

13. The apparatus as set forth in claim 1, further comprising at least one optical component to select one or more laser beam parameters of one or more of said laser diagnostic beams.

14. The apparatus as set forth in claim 13, wherein said optical component is a beam profiler, a collimator, a spherical element, an a-spherical element or a parabolic element.

15. The apparatus as set forth in claim 1, further comprising means to control each one of said n lasers.

16. The apparatus as set forth in claim 15, wherein said means to control comprises a single control panel.

17. The apparatus as set forth in claim 1, further comprising means to control one or more laser beam parameters of at least one of said laser diagnostic beams.

18. The apparatus as set forth in claim 1, wherein one or more of said n lasers is a gas laser, liquid laser, solid state laser, semiconductor diode laser, a tunable laser or a flashlight laser.

19. The apparatus as set forth in claim 1, wherein said means to deliver comprises a mirror-based optical delivery system to control said combined diagnostic beam.

20. The apparatus as set forth in claim 19, wherein said mirror-based optical delivery system has a spot size of 0.1 mm or less.

21. The apparatus as set forth in claim 19, wherein said mirror-based optical delivery device has a spot size of 0.1 mm or more.

22. The apparatus as set forth in claim 19, wherein said optical delivery device comprises linear delivery means.

23. The apparatus as set forth in claim 19, wherein said optical delivery device comprises three-dimensional delivery means.

24. The apparatus as set forth in claim 1, wherein said means to deliver comprises a micromanipulator.

25. The apparatus as set forth in claim 1, wherein said means to deliver comprises endoscopic delivery means.

26. The apparatus as set forth in claim 1, wherein said means to deliver comprises an optical device wherein said optical device comprising:
(a) n optical components aligned on an optical path to receive said laser diagnostic beams from said n lasers, wherein said laser nᵢ corresponds to said optical component nᵢ and i=1, . . . n, and wherein each of said n optical components directs and selectively combines said laser diagnostic beams of said n lasers along said optical path; and
(b) an optical delivery system connected to said optical path to deliver said combined diagnostic beam to said substance.

27. The apparatus as set forth in claim 26, wherein one or more of said n optical components is a wavelength selective mirror, a beam splitter or a wavelength selective filter.

28. The apparatus as set forth in claim 26, further comprising means to position said n optical components in said optical path or away from said optical path.

29. The apparatus as set forth in claim 26, further comprising position means to generate a subset of combinations of said laser diagnostic beams.

30. The apparatus as set forth in claim 1, further comprising means to enhance said fluorescence emission.

31. The apparatus as set forth in claim 30, wherein said means to enhance said fluorescence emission comprises an agent, a dye, a metallic particle or a fluorescent probe.

32. The apparatus as set forth in claim 1, wherein said substance is a biological tissue, a food, a fluid, a chemical compound, a biochemical compound, a bioengineering composition or a physical structure.

33. The apparatus as set forth in claim 1, wherein said diagnosis is a medical diagnosis and said laser diagnostic beams are medically useful diagnostic beams.

34. The apparatus as set forth in claim 1, further comprising means for treating said substance.

35. The apparatus as set forth in claim 34, wherein said means for treating said substance comprises a multiple laser treatment apparatus for simultaneous delivery of two or more laser treatment beams at said substance wherein each one of said laser treatment beams comprises at least one different laser beam parameter.

36. The apparatus as set forth in claim 1, wherein said apparatus is a handheld delivery apparatus.

37. The apparatus as set forth in claim 1, wherein said handheld delivery apparatus is a portable and transferable miniature handheld delivery apparatus with dimensions of 6″ by 12″ by 20″ or less.

38. The apparatus as set forth in claim 1, wherein said apparatus operates on independent power.

39. A multiple laser diagnostic apparatus, comprising:
(a) means to select two or more laser diagnostic beams selected for a diagnosis using fluorescence emission wherein each one of said laser diagnostic beams comprises at least one different laser beam parameter; and
(b) means to simultaneously deliver said laser diagnostic beams in a combined laser diagnostic beam at a substance at which said substance undergoes said fluorescence emission.

40. The apparatus as set forth in claim 39, wherein said laser beam parameters are wavelengths, fluences, power levels, energy levels, temporal parameters, geometrical parameters, spot sizes, linear delivery parameters or three-dimensional delivery parameters.

41. The apparatus as set forth in claim 39, further comprising means to detect said fluorescence emission.

42. The apparatus as set forth in claim 39, further comprising means to analyze said detected fluorescence emission.

43. The apparatus as set forth in claim 39, further comprising means to display said diagnosis.

44. The apparatus as set forth in claim 39, wherein said means to deliver comprises means to detect said fluorescence emission.

45. The apparatus as set forth in claim 44, wherein said means to detect comprises a plurality of optical paths.

46. The apparatus as set forth in claim 45, wherein said optical paths are optical fibers, articulated arms or waveguides.

47. The apparatus as set forth in claim 45, wherein one or more of said plurality of optical paths are positioned to detect a substantially uniform distribution of said fluorescence emission from said substance.

48. The apparatus as set forth in claim 39, wherein said means to select comprises at least one optical component to select one or more of said laser beam parameters of one or more of said laser diagnostic beams.

49. The apparatus as set forth in claim 39, wherein said means to select comprises means to control said laser diagnostic beams.

50. The apparatus as set forth in claim 39, wherein said means to deliver comprises a mirror-based optical delivery system to control said combined diagnostic beam.

51. The apparatus as set forth in claim 50, wherein said mirror-based optical delivery system has a spot size of 0.1 mm or less.

52. The apparatus as set forth in claim 50, wherein said mirror-based optical delivery device has a spot size of 0.1 mm or more.

53. The apparatus as set forth in claim 50, wherein said optical delivery device comprises linear delivery means.

54. The apparatus as set forth in claim 50, wherein said optical delivery device comprises three-dimensional delivery means.

55. The apparatus as set forth in claim 39, wherein said means to deliver comprises a micromanipulator.

56. The apparatus as set forth in claim 39, wherein said means to deliver comprises endoscopic delivery means.

57. The apparatus as set forth in claim 39, wherein said means to deliver comprises an optical device to combine said laser diagnostic beams.

58. The apparatus as set forth in claim 39, further comprising means for treating said substance.

59. The apparatus as set forth in claim 39, further comprising means for enhancing said fluorescence emission.

60. A method for diagnosing a substance, comprising the steps of:
(a) selecting two or more laser diagnostic beams selected for a diagnosis using fluorescence emission wherein each one of said laser diagnostic beams comprises at least one different laser beam parameter; and
(b) simultaneously delivering said laser diagnostic beams in a combined laser diagnostic beam at a substance at which said substance undergoes said fluorescence emission.

61. The method as set forth in claim 60, wherein said laser beam parameters are wavelengths, fluences, power levels, energy levels, temporal parameters, geometrical parameters, spot sizes, linear delivery parameters or three-dimensional delivery parameters.

62. The method as set forth in claim 60, wherein said step of selecting comprises the step of providing means to detect said fluorescence emission.

63. The method as set forth in claim 60, further comprising means to analyze said detected fluorescence emission.

64. The method as set forth in claim 60, further comprising means to display said diagnosis.

65. The method as set forth in claim 60, wherein said step of delivering comprises means to detect said fluorescence emission.

66. The method as set forth in claim 65, wherein said means to detect comprises a plurality of optical paths.

67. The method as set forth in claim 66, wherein said optical paths are optical fibers, articulated arms or waveguides.

68. The method as set forth in claim 66, further comprising the step of positioning one or more of said plurality of optical paths to detect a substantially uniform distribution of said fluorescence emission from said substance.

69. The method as set forth in claim 60, wherein said step of selecting comprises the step of providing at least one optical component to select one or more of said laser beam parameters of one or more of said laser diagnostic beams.

70. The method as set forth in claim 60, wherein said step of selecting comprises means to control said laser diagnostic beams.

71. The method as set forth in claim 60, wherein said step of delivering comprises the step of providing a mirror-based optical delivery system to control said combined diagnostic beam.

72. The method as set forth in claim 71, wherein said mirror-based optical delivery system has a spot size of 0.1 mm or less.

73. The method as set forth in claim 71, wherein said mirror-based optical delivery device has a spot size of 0.1 mm or more.

74. The method as set forth in claim 71, wherein said optical delivery device comprises linear delivery means.

75. The method as set forth in claim 71, wherein said optical delivery device comprises three-dimensional delivery means.

76. The method as set forth in claim 60, wherein said step of delivering comprises the step of providing a micromanipulator.

77. The method as set forth in claim 60, wherein said step of delivering comprises the step of providing endoscopic delivery means.

78. The method as set forth in claim 60, wherein said step of delivering comprises the step of providing an optical device to combine said laser diagnostic beams.

79. The method as set forth in claim 60, further comprising the step of providing means for treating said substance.

80. The method as set forth in claim 60, further comprising the step of providing means for enhancing said fluorescence emission.

81. A computer program to manage and control a simultaneous delivery of multiple laser diagnostic beams to a substance, comprising:
(a) means for selecting a diagnostic plan wherein said diagnostic plan comprises two or more laser diagnostic beams wherein each one of said laser diagnostic beams comprises at least one different laser beam parameter; and
(b) means for applying said diagnostic plan to said substance.

82. The computer program as set forth in claim 81, wherein said means for selecting comprises means for recommending said diagnostic plan.

83. The computer program as set forth in claim 81, wherein said means for selecting comprises a database of diagnostic plans.

84. The computer program as set forth in claim 81, wherein said means for selecting comprises means for comparing said diagnostic plan with a previous diagnostic plan.

85. The computer program as set forth in claim 81, further comprising means for entering data.

86. The computer program as set forth in claim 85, wherein said data comprises patient data, diagnostic plan data, maps, or complaint or disease data.

87. The computer program as set forth in claim 81, further comprising means for verifying said diagnostic plan.

88. The computer program as set forth in claim 81, further comprising communication means to communicate information between said computer program and one or more remote stations.

89. The computer program as set forth in claim 81, further comprising means to create a map based on said detected fluorescence emission.

90. The computer program as set forth in claim 89, wherein said map is a two-dimensional map.

91. The computer program as set forth in claim 89, wherein said map is a three-dimensional map.

92. The computer program as set forth in claim 81, further comprising means to create a database.

93. The computer program as set forth in claim 81, further comprising means to diagnose said substance.

94. The computer program as set forth in claim 93, further comprising means to recommend a treatment for said diagnosed substance, wherein said treatment comprises a laser treatment or a pharmaceutical treatment.

95. A database of a plurality of laser diagnostic plans wherein two or more laser diagnostic beams are delivered simultaneously to a substance, comprising:
(a) said plurality of diagnostic plans; and
(b) said one or more laser beam parameters for each one of said diagnostic plans wherein each one of said laser diagnostic beams comprises at least one different laser beam parameter.

96. The database as set forth in claim 95, wherein said diagnostic plans are medical diagnostic plans, chemical diagnostic plans, biochemical diagnostic plans, bioengineering diagnostic plans or physical diagnostic plans.

97. The database as set forth in claim 95, further comprising substance-related information.

98. The database as set forth in claim 95, further comprising patient-related information.

99. The database as set forth in claim 95, further comprising fluorescence emission maps.
